(19) United States
(12) Patent Application Publication
(10) Pub. No.: US 2007/0264234 A1
(43) Pub. Date: Nov. 15, 2007

(54) Cytokine Variant Polypeptides

(76) Inventors: Jon Sayers, Chesterfield (GB); Peter Artymiuk, Sheffield (GB); Richard Ross, Sheffield (GB)

Correspondence Address: KLARQUIST SPARKMAN, LLP, 121 SW SALMON STREET, SUITE 1600, PORTLAND, OR, 97204, US

(21) Appl. No.: 10/561,831

(22) PCT Filed: Jun. 28, 2004
(86) PCT No.: PCT/GB04/02827

§ 371 Date: Mar. 16, 2007

(30) Foreign Application Priority Data
Jun. 28, 2003 (GB) 0315182.6

(51) Int. Cl. A61K 48/00; A01K 67/027; A61K 31/7052; C07K 14/00; C07K 7/06; C12N 15/63; G01N 33/68; C12N 5/10; C12N 1/15; C07K 2/00; C07H 21/00; A61K 38/00

(52) U.S. Cl. 424/93.2; 435/243; 435/254.2; 435/320.1; 435/325; 435/419; 436/86; 514/2; 514/44; 530/300; 530/324; 530/328; 530/330; 536/23.1; 800/14

(57) ABSTRACT

We describe modified cytokine ligand polypeptides comprising a modified amino acid sequence which is a modification of the native cytokine amino acid sequence of said ligand, wherein the native amino terminal and carboxyl terminal amino acid residues of the native polypeptide are linked, directly or indirectly, together, characterised in that said ligand is provided with alternative amino terminal and carboxyl terminal amino acid residues and further wherein at least one binding domain for said ligand's cognate binding partner or receptor complex is disrupted.

The invention relates to polypeptides comprising cytokine ligands wherein at least one binding site for said ligand's cognate receptor is disrupted; oligomers thereof and their use as pharmaceutical agents.

A large group of growth factors, referred to as cytokines, are involved in a number of diverse cellular functions. These include, by example and not by way of limitation, modulation of the immune system, regulation of energy metabolism and control of growth and development. Cytokines mediate their effects via receptors expressed at the cell surface on target cells. Cytokine receptors can be divided into four separate sub groups. Type 1 (growth hormone (GH) family) receptors are characterised by four conserved cysteine residues in the amino terminal part of their extracellular domain and the presence of a conserved Trp-Ser-Xaa-Trp-Ser motif in the C-terminal part. The repeated Cys motif are also present in Type 2 (interferon family) and Type III (tumour necrosis factor family).

It is known that many cytokine ligands interact with their cognate receptor via specific sites. Some cytokine receptors have both high affinity ligand binding sites and low affinity binding sites.

For example, it is known that a single molecule of GH associates with two receptor molecules (GHR) (Cunningham et al, 1991; de Vos et al., 1992; Sundstrom et al., 1996; Clackson et al., 1998). This occurs through two unique receptor-binding sites on GH and a common binding pocket on the extracellular domain of two receptors. Site 1 on the GH molecule has, a higher affinity than site 2, and receptor dimerization is thought to occur sequentially with one receptor binding to site 1 on GH followed by recruitment of a second receptor to site 2. The extracellular domain of the GHR exists as two linked domains each of approximately 100 amino acids. It is a conformational change in these two domains that occurs on hormone binding with the formation of the trimeric complex GHR-GH-GHR. Internalisation of the GHR-GH-GHR complex is followed by a recycling step whereby the receptor molecule is regenerated for further use within the cell.

A variety of different stoichiometries are employed by different cytokines and other ligands on receptor binding. Thus erythropoetin, like GH, forms a trimeric receptor-hormone-receptor complex. Interleukin-4 forms a trimeric receptor-hormone-different receptor complex. Other cytokines, for example leptin and GCSF, form tetrameric receptor-hormone-hormone-receptor complexes, and others (eg interleukin 6) probably form hexameric complexes consisiting of two soluble receptor molecules, two transmembrane receptor molecules and two cytokine molecules. In each case there is a primary high affinity binding site that locates the cytokine to the receptor complex, and additional sites which play secondary roles in altering the conformation or recruiting other molecules and thereby initiating signalling.

Variant cytokine polypeptides are known. For example, GH variants are disclosed in U.S. Pat. No. 5,849,535. The modification to GH is at both site 1 and site 2 binding sites. The modifications to site I produce a GH molecule that has a higher affinity for GHR compared to wild-type GH. These modified GH molecules act as agonists. There is also disclosure of site 2 modifications that result in the creation of GH antagonists. Further examples of modifications to GH which alter the binding affinity of GH for site 1 are disclosed in U.S. Pat. No. 5,854,026; U.S. Pat. No. 6,004,931; U.S. Pat. No. 6,022,711; U.S. Pat. No. 6,057,292; and U.S. Pat. No. 6,136,563. These modifications relate to point mutations at specific positions in GH which produce a molecule with altered signalling properties.

Circular permutation is a means to generate polypeptide variants that retain the overall tertiary structure of a native polypeptide but re-orders the primary linear sequence by forming new amino and carboxyl termini. The process generates molecules with altered biological properties. The process includes the fusion of the natural amino and carboxyl termini either directly or by using linker molecules that are typically peptide linkers. The conceptually circularised molecule is then cut to generate new amino and carboxyl termini. Circularly permuted polypeptides can be generated either recombinantly or by in vitro peptide synthesis.

Circular permutation has been used to generate chimeric molecules with altered biological activity.

For instance, WO95/27732 discloses the creation of a circularly permuted IL-4 ligand fused to a cytotoxic agent. The permuted IL-4-agent has altered affinity and cytotoxicity when compared to a native IL-4-agent and has efficacy with respect to killing cancer cells which are exposed to the conjugated polypeptide.

WO99/51632 describes the use of circular permutation to generate novel streptavidin binding proteins that have reduced affinity for biotin. The circularly permuted streptavidin is fused to a second polypeptide to create a fusion protein that differentially binds biotin. The reduced affinity of the strepavidin fusion protein for biotin facilitates release of the fusion protein when biotin is used as a drug delivery vehicle.

WO01/51629 discloses circularly permuted bacterial β-lactamase and its use as a marker protein for the detection of interactions between intracellular and extracellular proteins that assemble with the permuted polypeptide.

Methods to identify circularly permuted polypeptides are also known. For example, WO00/18905, which is incorporated by reference, describes a method to identify permuted polypeptides, referred to as “permuteins”, using a phage display vector into which a library of permuted genes are inserted. The expression of the library at the surface of the display vector is detected by exposure of the expressed library to a binding protein that potentially interacts with a permutein.

WO01/30998, which is incorporated by reference, discloses a further method to generate and identify circularly permuted proteins. The invention relates to the formation of fusion proteins comprising the amino terminal part of a first protein fused to the carboxyl terminal part of a different second protein from which permuteins are synthesised. A library of fusion proteins is created which can be screened by phage display.

We have applied the process of circular permutation to cytokine ligands to generate polypeptide variants that are “gapped” at binding sites for binding partners and/or their receptors to produce ligands with altered properties (antagonists or agonists). Ligands that interact with receptors to bring about a suitable biochemical response are known as agonists and those that prevent, or hinder, a biochemical response are known as antagonists. For example, and not by way of limitation, cell specific growth factors are ligands that act as agonists and bind receptors located in cell membranes to activate cell division, growth or differentiation.

As an illustration of the technique, we have generated a series of circularly permuted GH constructs in which the formation of new amino and carboxyl terminal termini is localised to site 2 of GH thereby disrupting the low affinity binding site for ligand binding to the GHR. This therefore allows docking of GH via its high affinity site 1 domain but produces a complex which is incapable of activating GHR.

We also disclose oligomers (tandems, trimers, etc) of said permuted polypeptides that have additional properties, for example delayed clearance from the circulatory system. The in vivo efficacy of many cytokines, for example GH, is determined in part by the affinity for GHR and rate of clearance from the circulation. Permuted polypeptides may also be fused to the extracellular binding domain of their cognate receptor(s). For example, we describe in PCT/GB01/02645; WO01/096565, the fusion of ligand binding domains to their cognate receptors (for example the fusion of GH to GHR via linker molecules) that retain biological activity and have the advantageous property of delayed clearance.

The kidneys are relatively small organs that receive approximately 25% of cardiac output. The kidneys perform several important functions primarily related to the regulation of the composition and volume of body fluids. The kidneys filter about 100 litres of plasma every day and of the blood flow in and out of a kidney only approximately 1% becomes urine. Approximately 20% of the plasma that passes through the kidney gets filtered into the nephron. Filtration takes place. in the glomerulus that is driven by the hydrostatic pressure of the blood. Water and small molecules are filtered whereas blood cells and large molecules, for example polypeptides, do not pass through the glomerular filter. Those polypeptides with an effective molecular weight above 70 kDa are not cleared by glomerular filtration because they are simply too large to be filtered. Certain proteins of small molecular weight are filtered by the glomerulus and are found in the urine. For example, GH has a molecular weight of 22.1 kDa and the kidney is responsible for clearing up to 60-70% of GH in humans (Baumann, 1991; Haffner et al, 1994), and up to 67% in rat (Johnson & Maack, 1977). Other examples of relatively small molecular weight polypeptides that are filtered by the kidney include leptin, erythropoeitin, and IL-6.

According to an aspect of the invention there is provided a modified ligand polypeptide comprising a modified amino acid sequence which is a modification of the native amino acid sequence of said ligand, wherein the native amino terminal and carboxyl terminal amino acid residues of the native polypeptide are linked, directly or indirectly, together, characterised in that said ligand is provided with alternative amino terminal and carboxyl terminal amino acid residues and further wherein at least one binding domain for said ligand's cognate binding partner is disrupted.

According to an aspect of the invention there is provided a modified cytokine ligand polypeptide comprising a modified amino acid sequence which is a modification of the native cytokine amino acid sequence of said ligand, wherein the native amino terminal and carboxyl terminal amino acid residues of the native polypeptide are linked, directly or indirectly, together, characterised in that said ligand is provided with alternative amino terminal and carboxyl terminal amino acid residues and further wherein at least one binding domain for said ligand's binding partner is disrupted.

In a preferred embodiment of the invention said native cytokine ligand is selected from the group consisting of: growth hormone; leptin; erythropoietin; prolactin; tumour necrosis factor (TNF), interleukins (IL), IL-2, IL-3, IL-4, IL-5, IL-6, IL-7, IL-9, IL-10, IL-11; the p35 subunit of IL-12, IL-13, IL-15; granulocyte colony stimulating factor (G-CSF); granulocyte macrophage colony stimulating factor (GM-CSF); ciliary neurotrophic factor (CNTF); cardiotrophin-1 (CT-1); leukemia inhibitory factor (LIF); oncostatin M (OSM); interferon, IFNα and IFNγ, osteoprotogerin (OPG).

In a further preferred embodiment of the invention said binding partner is a receptor for said ligand.

In an alternative preferred embodiment of the invention said binding partner is a second ligand which forms a complex with said ligand and/or said receptor.

In a preferred embodiment of the invention said cytokine ligand is growth hormone.

In a further preferred embodiment of the invention said native amino terminal and carboxyl terminal amino acid residues are directly linked to each other.

In an alternative preferred embodiment of the invention said native amino terminal and carboxyl terminal amino acid residues are indirectly linked by a linking molecule. Preferably said linking molecule is a peptide linkage.

In a preferred embodiment of the invention said linking peptide is a flexible peptide linker.

Preferably the flexible linker is a polypeptide that comprises 5 to 30 amino acid residues. More preferably the linker comprises 10 to 20 amino acid residues. More preferably still the linker comprises at least one copy of the peptide:
Gly Gly Gly Gly Ser (hereinafter referred to as “Gly4Ser”).

In one embodiment of the invention the linker is 10 amino acids in length and comprises two copies of the Gly4Ser linker. In an alternative embodiment of the invention, the linker is 15 amino acids in length and comprises three copies of the Gly4Ser linker. In yet an alternative embodiment, the linker is 20 amino acids in length and comprises four copies of the Gly4Ser linker.

In an alternative embodiment of the invention said linker is an inflexible linker, for example a linker wherein said linker is, over part of its length, has a α-helical region.

For example, Arai et al (Protein Eng 14(8): 529-532 (2001), which is incorporated by reference, investigated the use of helix-forming peptides A(EAAAK)ₙA to separate domains of different green fluorescent proteins and measured the separation of the proteins using FRET, showing such linkers to behave as rigid entities of fixed length. JP2002247997, which is incorporated by reference, utilises an a-helical linker sequence to link IL-6 ligand to IL-6 receptor. It is envisaged that linkers that have properties between the extremes of a flexible linker and a helical linker may also be utilised to link respective native ends of cytokine ligands.

In a further preferred embodiment of the invention said receptor binding domain of said ligand comprises a low affinity bind site.

In a preferred embodiment of the invention said low affinity binding domain is site 2 of growth hormone.

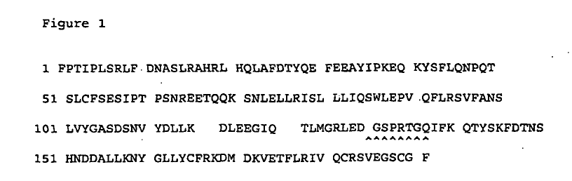

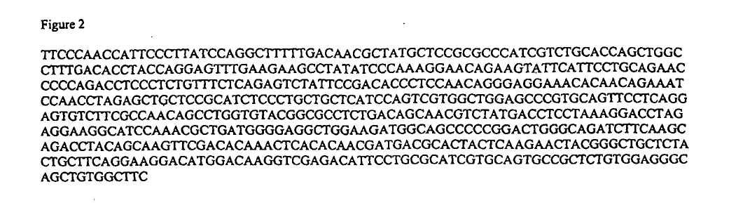

In a further preferred embodiment of the invention said low affinity binding domain of growth hormone is between about amino acid 116—amino acid 122 of human growth hormone as represented by the amino acid sequence shown in FIG. 1.

In a further preferred embodiment of the invention said alternative amino terminal and carboxyl terminal amino acid residues are derived from between amino acid 116 and amino acid 122 of human growth hormone as represented by FIG. 1.

In a further preferred embodiment of the invention said alternative amino terminal and carboxyl terminal amino acid residues are derived from between amino acid 118 and amino acid 121 of human growth hormone as represented by FIG. 1.

In a further preferred embodiment of the invention said alternative amino terminal and carboxyl terminal amino acid residues are derived from between amino acid 119 and amino acid 121 of human growth hormone as represented by FIG. 1.

In a further preferred embodiment of the invention said alternative amino terminal and carboxyl terminal amino acid residues are derived from between amino acid 120 and amino acid 121 of human growth hormone as represented by FIG. 1.

In a further preferred embodiment of the invention said alternative amino terminal and carboxyl terminal amino acid residues are derived from between amino acid 118 and amino acid 120 of human growth hormone as represented by FIG. 1.

In a further preferred embodiment of the invention said alternative amino terminal and carboxyl terminal amino acid residues are derived from between amino acid 119 and amino acid 120 of human growth hormone as represented by FIG. 1.

In an alternative preferred embodiment of the invention said alternative amino terminal and carboxyl terminal amino acid residues are derived from between about amino acid 100 and amino acid 102 of human growth hormone as represented by the amino acid sequence shown in FIG. 1

In a further alternative preferred embodiment of the invention alternative amino terminal and carboxyl terminal amino acid residues are derived from between about amino acid 130—amino acid 132 of human growth hormone as represented by the amino acid sequence shown in FIG. 1

In a further preferred embodiment of the invention there is provided an oligomeric cytokine ligand polypeptide comprising at least two modified cytokine ligand polypeptides according to the invention wherein said ligands are linked, either directly or indirectly, together.

In a preferred embodiment of the invention said ligands are linked by a flexible peptide linker molecule. In an alternative preferred embodiment of the invention said ligands are linked by an inflexible peptide linker molecule, preferably said linker molecule comprises a α-helical region.

In a preferred embodiment of the invention said oligomer comprises two modified cytokine ligand polypeptides.

In a further preferred embodiment of the invention said said oligomer comprises, at least 3; 4; 5; 6; 7; 8; 9; or at least 10 modified cytokine ligand polypeptides.

In a further preferred embodiment of the invention said oligomeric cytokine polypeptide comprises at least two modified growth hormone polypeptides as hereindescribed. Preferably said oligomeric growth hormone polypeptide comprises multiple ligand polypeptides.

In an alternative preferred embodiment of the invention there is provided an oligomeric cytokine ligand polypeptide comprising at least one modified cytokine ligand polypeptide according to the invention linked, either directly or indirectly, to at least one native cytokine ligand polypeptide from which said modified cytokine ligand polypeptide was derived.

In a further alternative embodiment of the invention there is provided a modified cytokine ligand polypeptide according to the invention linked to at least one extracellular ligand binding domain of said ligand's cognate receptor.

In our co-pending application, WO01/096565, which is incorporated by reference, we disclose fusion proteins which translationally fuse the ligand binding domain of a cytokine to the extracellular receptor binding domain of said ligand via flexible peptide linkers. These fusion proteins have delayed clearance and agonist activity. The fusion of cytokine to cognate receptor provides an immunologically silent polypeptide which has a molecular weight which slows renal clearance. Modified cytokine ligand polypeptides as hereindisclosed could also benefit from delayed clearance.

Peptide linkers that link cytokine ligand polypeptides to one another to form oligomeric polypeptides (dimers, trimers etc) and to cognate extracellular receptor binding domains are either flexible (e.g. Gly4Ser) or inflexible (e.g. α-helical) or intermediate (e.g. a combinational linker which is part helical) as described above. Linkers may also contain cleavage sites to provide oligomeric polypeptides with delayed release characteristics.

In a preferred embodiment of the invention said linker comprises a cleavage site, preferably a proteolytic cleavage site.

Preferably said cleavage site is sensitive to a serum protease or a matrix metalloprotease.

In a preferred embodiment of the invention said cleavage site comprises the amino acid sequence: LVPRGS, or variant thereof.

Preferably, said cleavage site comprises the amino acid sequence PGI(S), or variant thereof.

More preferably still said cleavage site comprises the amino acid sequence: LVPRGS PGI, or variant thereof.

Alternatively, said cleavage site comprises at least two copies of the amino acid sequence GGGGS, or functional variant thereof, which flank said cleavage site.

In a further preferred embodiment of the invention said cleavage site is sensitive to the serum protease thrombin.

According to a further aspect of the invention there is provided a nucleic acid molecule which encodes a modified cytokine ligand polypeptide or an oligomeric modified cytokine ligand polypeptide according to the invention.

According to a further aspect of the invention there is provided a vector comprising a nucleic acid molecule according to the invention.

In a preferred embodiment of the invention said vector is an expression vector adapted for eukaryotic gene expression.

Typically said adaptation includes, the provision of transcription control sequences (promoter/enhancer sequences) which mediate cell/tissue specific expression. These promoter sequences may be cell/tissue specific, inducible or constitutive.

Promoter is an art recognised term and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences and are therefore position independent). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to a number of environmental cues that include, by example and not by way of limitation, intermediary metabolites (e.g. glucose, lipids), environmental effectors (e.g. heat).

Promoter elements also include so called TATA box and RNA polymerase initiation selection (RIS) sequences which function to select a site of transcription initiation. These sequences also bind polypeptides that function, inter alia, to facilitate transcription initiation selection by RNA polymerase.

Adaptations also include the provision of selectable markers and autonomous replication sequences which both facilitate the maintenance of said vector in either the eukaryotic cell. Vectors which are maintained autonomously are referred to as episomal vectors.

Adaptations which facilitate the expression of vector encoded genes include the provision of transcription termination/polyadenylation sequences. This also includes the provision of internal ribosome entry sites (IRES) that function to maximise expression of vector encoded genes arranged in bicistronic or multi-cistronic expression cassettes.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc. (1994).

It will be apparent to one skilled in the art that the vectors according to the invention could be gene therapy vectors. Gene therapy vectors are typically virus based. A number of viruses are commonly used as vectors for the delivery of exogenous genes. Commonly employed vectors include recombinantly modified enveloped or non-enveloped DNA and RNA viruses, preferably selected from baculoviridiae, parvoviridiae, picornoviridiae, herpesveridiae, poxviridae, adenoviridiae, or picornnaviridiae. Chimeric vectors may also be employed which exploit advantageous elements of each of the parent vector properties (See e.g., Feng, et al. (1997) Nature Biotechnology 15:866-870). Such viral vectors may be wild-type or may be modified by recombinant DNA techniques to be replication deficient, conditionally replicating or replication competent.

Preferred vectors are derived from the adenoviral, adeno-associated viral and retroviral genomes. In the most preferred practice of the invention, the vectors are derived from the human adenovirus genome. Particularly preferred vectors are derived from the human adenovirus serotypes 2 or 5. The replicative capacity of such vectors may be attenuated (to the point of being considered “replication deficient”) by modifications or deletions in the E1a and/or E1b coding regions. Other modifications to the viral genome to achieve particular expression characteristics or permit repeat administration or lower immune response are preferred.

Alternatively, the viral vectors may be conditionally replicating or replication competent. Conditionally replicating viral vectors are used to achieve selective expression in particular cell types while avoiding untoward broad spectrum infection. Examples of conditionally replicating vectors are described in Pennisi, E. (1996) Science 274:342-343; Russell, and S. J. (1994) Eur. J. of Cancer 30A(8): 1165-1171.

Additional examples of selectively replicating vectors include those vectors wherein a gene essential for replication of the virus is under control of a promoter which is active only in a particular cell type or cell state such that in the absence of expression of such gene, the virus will not replicate. Examples of such vectors are described in Henderson, et al., U.S. Pat. No. 5,698,443 issued Dec. 16, 1997 and Henderson, et al., U.S. Pat. No. 5,871,726 issued Feb. 16, 1999 the entire teachings of which are herein incorporated by reference. Vectors may also be non-viral and are available from a number of commercial sources readily available to a person-skilled in the art. For example, the vectors may be plasmids that can be episomal or integrating.

According to a yet further aspect of the invention there is provided a cell transfected or transformed with a nucleic acid molecule or vector according to the invention.

In a preferred embodiment of the invention said cell is a eukaryotic cell. Preferably said cell is selected from the group consisting of: a mammalian cell (e.g. Chinese Hamster Ovary cell); yeast cells (e.g. Saccharomyces spp, Pichia spp); insect cells (e.g. Spodoptera spp) or plant cells.

According to a yet further aspect of the invention there is provided a non-human transgenic mammal transfected/transformed with the nucleic acid molecule or vector according to the invention.

According to a yet further aspect of the invention there is provide a modified cytokine ligand polypeptide, an oligomeric modified cytokine ligand polypeptide, a nucleic acid molecule, a vector or a cell according to the invention for use as a pharmaceutical.

Preferably said ligand is an antagonist. In an alternative embodiment of the invention said ligand is an agonist.

Preferably said polypeptide, nucleic acid molecule, vector or cell is used in a pharmaceutical composition.

When administered the pharmaceutical composition of the present invention is administered in pharmaceutically acceptable preparations. Such preparations may routinely contain pharmaceutically acceptable concentrations of salt, buffering agents, preservatives, compatible carriers, and optionally other therapeutic agents.

The pharmaceutical composition of the invention can be administered by any conventional route, including injection. The administration and application may, for example, be oral, intravenous, intraperitoneal, intramuscular, intracavity, intra-articuar, subcutaneous, topical (eyes), dermal (e.g a cream lipid soluble insert into skin or mucus membrane), transdermal, or intranasal.

Pharmaceutical composition of the invention is administered in effective amounts. An “effective amount” is that amount of a pharmaceutical/composition that alone, or together with further doses or synergistic drugs, produces the desired response. This may involve only slowing the progression of the disease temporarily, although more preferably, it involves halting the progression of the disease permanently. This can be monitored by routine methods or can be monitored according to diagnostic methods.

The doses of the pharmaceutical composition administered to a subject can be chosen in accordance with different parameters, in particular in accordance with the mode of administration used and the state of the subject (i.e. age, sex). When administered, the pharmaceutical composition of the invention is applied in pharmaceutically-acceptable amounts and in pharmaceutically-acceptable compositions. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical composition may be combined, if desired, with a pharmaceutically-acceptable carrier. The term “pharmaceutically-acceptable carrier” as used herein means one or more compatible solid or liquid fillers, diluents or encapsulating substances that are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction that would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous or non-aqueous preparation that is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

Polypeptides/nucleic acid molecules etc according to the invention can be incorporated into liposomes. Liposomes are lipid based vesicles which encapsulate a selected therapeutic agent which is then introduced into a patient. The liposome is manufactured either from pure phospholipid or a mixture of phospholipid and phosphoglyceride. Typically liposomes can be manufactured with diameters of less than 200 nm, this enables them to be intravenously injected and able to pass through the pulmonary capillary bed. Furthermore the biochemical nature of liposomes confers permeability across blood vessel membranes to gain access to selected tissues. Liposomes do have a relatively short half-life. So called STEALTH^{R} liposomes have been developed which comprise liposomes coated in polyethylene glycol (PEG). The PEG treated liposomes have a significantly increased half-life when administered intravenously to a patient. In addition, STEALTH^{R} liposomes show reduced uptake in the reticuloendothelial system and enhanced accumulation selected tissues. In addition, so called immuno-liposomes have been develop which combine lipid based vesicles with an antibody or antibodies, to increase the specificity of the delivery of the agent to a selected cell/tissue.

The use of liposomes as delivery means is described in U.S. Pat. No. 5,580,575 and U.S. Pat. No. 5,542,935.

According to a further aspect of the invention there is provided a screening method to generate modified cytokine ligand polypeptides according to the invention comprising the steps of:
i) forming a preparation comprising native cytokine ligand polypeptide molecules wherein the native amino terminal and carboxyl terminal amino acids are linked either directly or indirectly together,
ii) generating modified cytokine ligand polypeptide molecules wherein said molecules have alternative amino terminal and carboxyl terminal amino acids; and
iii) testing the activity of said modified cytokine ligand polypeptides.

In a preferred method of the invention said native cytokine is growth hormone.

Bioassays to test the activity of, for example growth hormone are known in the art and are disclosed in WO01/096565.

According to a further aspect of the invention there is provided a modified cytokine ligand polypeptide identified by the method according to the invention.

In a preferred embodiment of the invention said modified cytokine ligand polypeptide is growth hormone.

According to a yet further aspect of the invention there is provided a method of treatment of an animal, preferably a human, comprising administering an effective amount of a nucleic acid and/or vector and/or polypeptide and/or cell according to the invention.

An embodiment of the invention will now be described by example only and with reference to the following figures:

FIG. 1 shows the amino acid sequence of human GH with binding site for GHR indicated with arrows;

FIG. 2 shows the nucleic acid sequence of human GH;

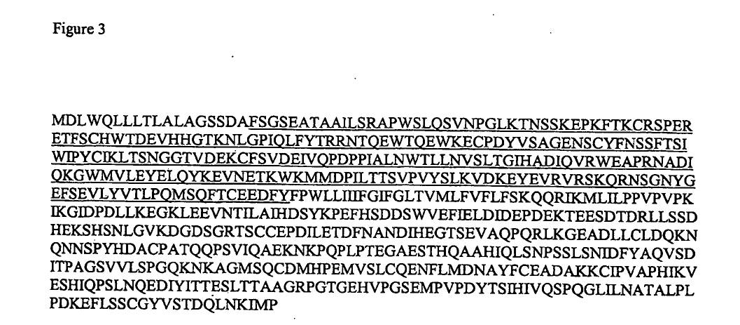

FIG. 3 shows the amino acid sequence of GHR (extracellular domain underlined);

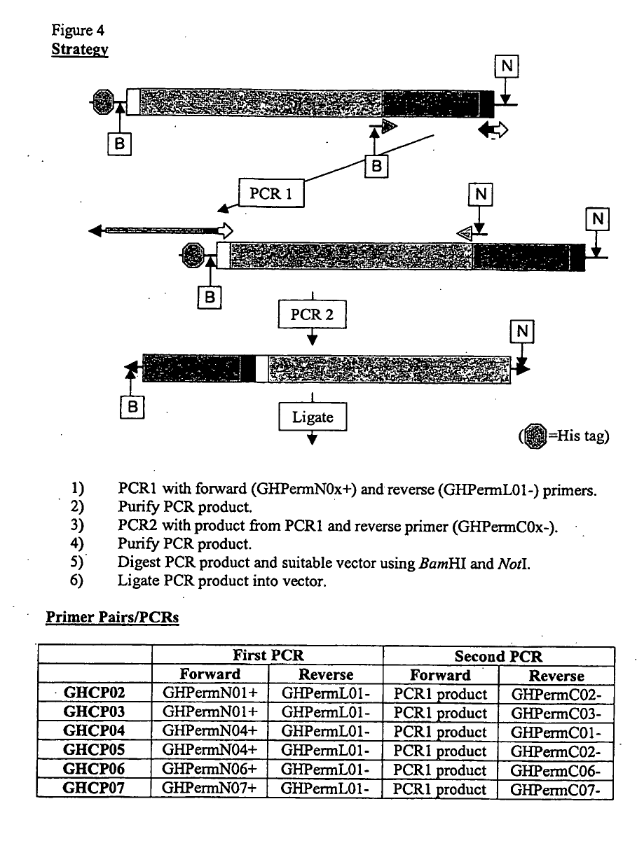

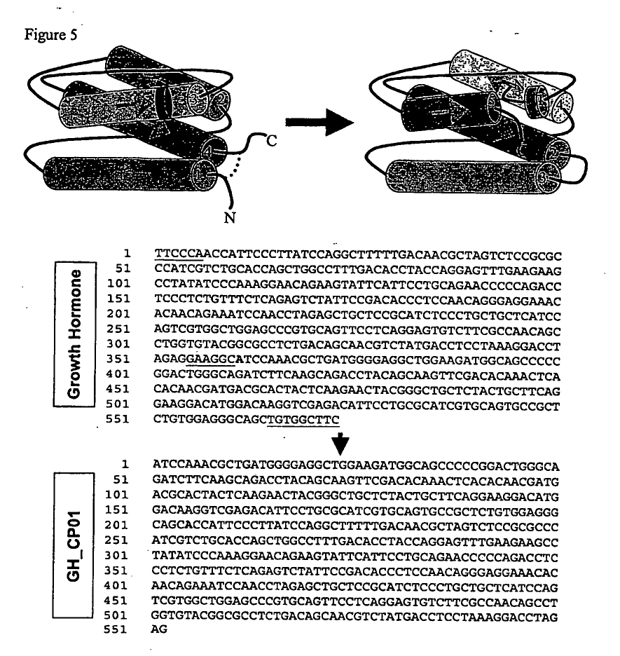

FIG. 4 Cloning strategy for the circular permutation of Growth Hormone. In the first PCR reaction a forward primer (GH_CPFor) and a linker primer (GH_CPLink) are used to produce a ‘megaprimer’. This megaprimer is used with a reverse primer (GH_CPRev) to generate the circularly permutated GH gene. Appropriate restriction sites [BamHI (B) and NotI (N)] are engineered into the forward and reverse primers to facilitate ligation into the vector pTrcHis-TOPO;

FIG. 5: Schematic and DNA sequence showing the strategy used to generate GH_CP01. (A) a schematic diagram showing how GH is transformed into GH_CP01; Glu120 (grey disc) is removed by initiating the new gene at residue 121 and terminating the protein at residue 118, the ‘old’ termini are linked by joining the termini to make a 6 amino acid linker. The helices are numbered in order (from N to C terminus) and the arrows denote the direction of the helices (from N to C). (B) the DNA sequence of GH and GH_CP01; the nucleotides removed from GH to produce GH_CP01 are underlined, the initiation nucleotide for GH_CP01 in GH, and vice versa, are shown in bold;

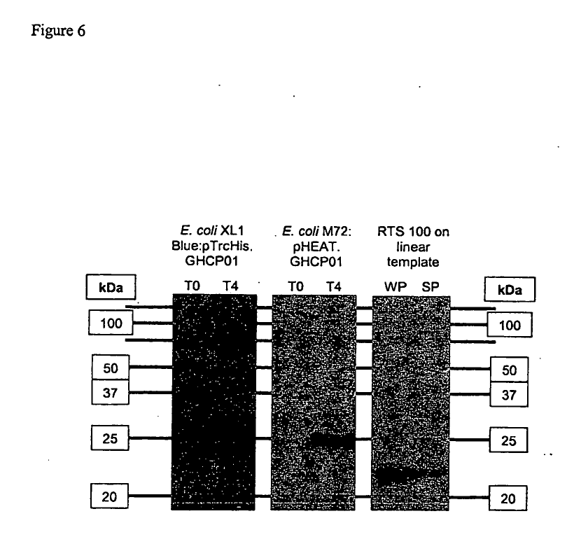

FIG. 6: Western blots showing the expression of GH_CP01 in three different systems. The blots show that GH_CP01 is expressed and detected by the antibody probes used in the western blot system utilised. T0 shows expression at the time of induction, T4 shows expression 4 hours post-induction, WP shows whole protein in the RTS reaction and SP shows only soluble protein in the RTS reaction. Protein produced from linear template lacks N-terminal His tag and adapter sequence and hence migrates slightly faster;

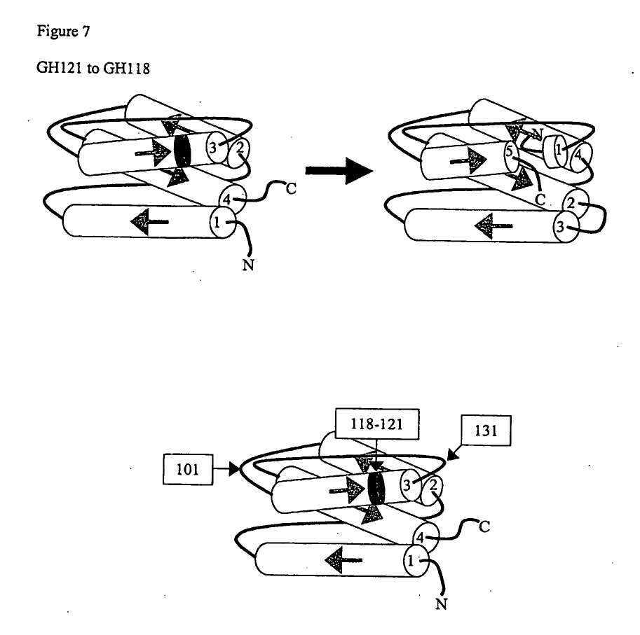

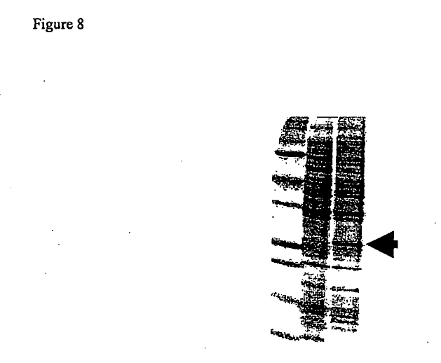

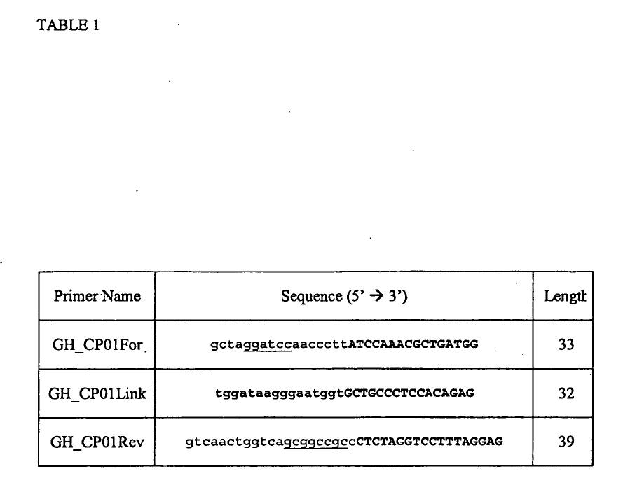

FIG. 7 is a diagrammatic representation of circularly permuted GH molecules;

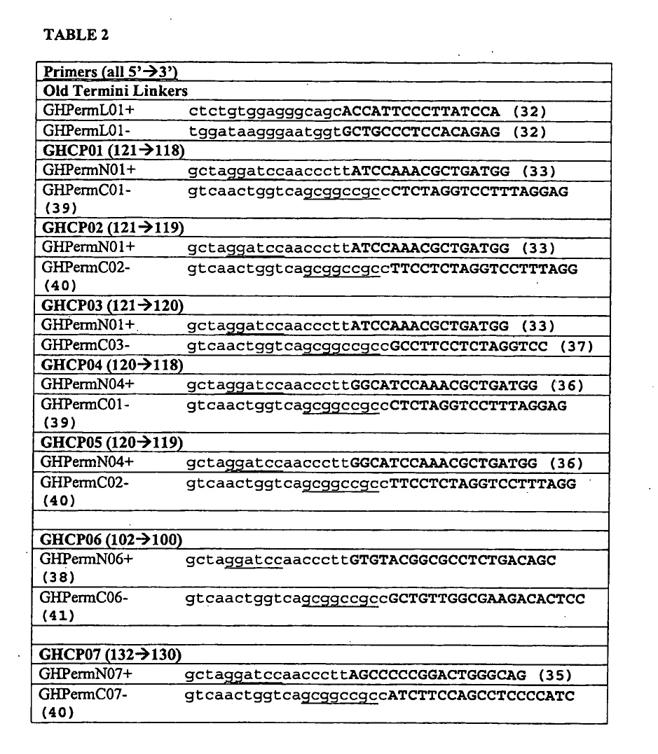

FIG. 8 illustrates expression of CP02 protein in E. coli. The induced band of CP02 protein is denoted by the arrowhead. Lane 1: BioRad protein marker; Lane 2: Non-induced cells carrying GHCP02 construct in LB media; Lane 3: Induced cells carrying GHCP02 construct;

Table 1 Primers used to generate GH_CP01. The bold characters denote sequence that anneals to the GH gene; the underlined characters denote endonuclease restriction sites (BamHI—ggatcc; NotI—gcggccgc). In the linker primer, GH_CP01Link, the sequence that anneals to the carboxyl terminus of GH is shown in UPPER-CASE; and

Table 2 Primers used to generate further circularly permuted GH molecules.

Materials and Methods

Growth hormone was circularly permutated by using a 2-step PCR methodology. The first PCR reaction generated a DNA fragment encoding the new amino terminus to the end of the GH gene and an overhang which could anneal to the start of the GH gene. This PCR product was then used as a ‘megaprimer’ in another PCR reaction to generate the full length GH_CP gene. The relevant primers were designed with restriction digestion sites for ligation into an appropriate vector (FIG. 4).

The GH_CP gene was ligated into an expression vector and this transformed into an appropriate strain of E. coli. Expression of GH_CP was confirmed by western blot of protein from induced cultures of E. coli containing the GH_CP expression plasmid, the blots were probed using mouse anti-GH antibodies (10A7, mouse IgG1) and Sheep anti-mouse-HRP antibodies (Amersham).

The GH_CP protein was purified from cell lysates using a metal chelate affinity column (Probond resin, Invitrogen) followed by an ion exchange column (MonoQ, Pharmacia).

Generation of the GH CP01 Gene

The first embodiment of the growth hormone circular permutation is GH_CP01. The amino terminus of this construct initiated at residue Ile121 of GH and the carboxy terminus was at residue Glu118 of GH. The ‘old’ termini of GH were linked by a 6 amino acid linker, which was formed by joining the ‘old’ termini −3 amino acids from the first helix at the amino terminus and +3 residues from the last helix at the carboxy terminus (FIG. 5)

Two PCR reactions were required to generate the circularly permutated GH gene. Three primers were designed (Table 1), GH_CP01For consisted of a BamHI restriction endonuclease site followed by the DNA sequence of the new amino terminus of the permutated GH; GH_CP01Rev was an antisense primer consisting of the DNA sequence of the new carboxy terminus of the permutated GH and a NotI restriction endonuclease site; GH_CP01Link was an antisense primer designed to anneal to both termini of GH.

The primers GH_CP01For and GHCP01Link and the template pTrcHisGHstop were used in the first PCR reaction to produce a ‘megaprimer’. This megaprimer was purified from the PCR reaction and then used in conjunction with GHCP01Rev, again using pTrcHisGHstop as the template, to produce the full length gene for the circularly permutated GH (GH_CP01). This gene was then ligated into the pTrcHis-TOPO plasmid vector between the BamHI and NotI restriction endonuclease sites. The vector was then transformed into E. coli XL1 Blue cells. The success of the circular permutation of GH was confirmed by DNA sequencing of the pTrcHis.GH_CP01 plasmid.

Expression of the GH CP

A variety of expression systems were used to optimise the production of GH_CP01. Expression was carried out from the E. coli XL1 Blue:pTrcHis.GH_CP01 cells induced with IPTG. The GH_CP01 gene was also subcloned into the pHEAT vector and the resulting E. coli M72:pHEAT.GH_CP01 cells induced by thermal regulation. Cell free in vitro translation was also used for protein expression; both linear template and the gene subcloned into the pIVEX-23MCS vector were used to express GH_CP01 in the RTS system (Roche). All these systems produced soluble protein (FIG. 6).

Purification of GH CP

GH_CP01 protein was purified from E. coli XL1 Blue:pTrcHis.GH_CP01. The cells were harvested from the induced growth cultures were resuspended in Resuspension Buffer (20 mM sodium phosphate buffer, 500 mM sodium chloride, 5% glycerol, 25 μg/ml PMSF, pH 7.8) were lysed by a 30 minute incubation on ice after the addition of 100 μg/ml (final concentration) hen egg white lysozyme, 250 μg/ml (final concentration) of sodium deoxycholate was then added and the solution incubated for a further 30 minutes on ice, the lysed cells were then sonicated. Insoluble material was removed by centrifugation at 1900 rpm for 30 minutes.

The cleared cell lysate was applied to a 5 ml Probond resin column (Invitrogen) which had been charged with Co²⁺ and equilibrated with Equilibration Buffer (20 mM sodium phosphate buffer, 500 mM sodium chloride, 5% glycerol, pH 7.8), after loading the protein sample the column was washed with a further 10-20 ml of Equilibration Buffer. The column was then washed with Wash Buffer (20 mM sodium phosphate buffer, 500 mM sodium chloride, 5% glycerol, pH 6.0) until the OD₂₈₀ of the eluate <0.01. Bound protein was eluted using 5 ml Elution Buffer (Wash Buffer containing 500 mM imidazole, pH 6.0).

The protein was dialysed overnight against Low Salt Buffer (25 mM TRIS, 1 mM EDTA, 5% glycerol, pH 8.0) and then centrifuged to remove any particulate matter. The protein sample was then loaded onto a Mono-Q column (Pharmacia), which had been pre-equilibrated with Low Salt Buffer. After a 10 column volume wash with Low Salt Buffer, the bound proteins were eluted over 20 column volumes using a gradient between 0M sodium chloride to 1M sodium chloride (in 25 mM TRIS, 1 mM EDTA, 5% glycerol, pH 8.0). Peaks on the elution profile were analysed by SDS-PAGE and western blotting.

GH_CP protein was then concentrated (if required) using an Amicon Centriprep Y-10 column.

The purity of the purified GH_CP01 was confirmed by SDS-PAGE, by both coomassie staining and western blot (results not shown). Once the integrity of this sample had been confirmed, GH_CP01 was submitted to the previously established bioassay (Ross et al., 1997).

### EXAMPLE 1

Alternative approach to constructing circular permutations by way of example:

The circular permuted hGH described earlier

IQTLMGRLED GSPRTGQIFK QTYSKFDTNS HNDDALLKNY
GLLYCFRKDM DKVETFLRIV QCRSVEGSTI PLSRLFDNAS
LRAHRLHQLA FDTYQEFEEA YIPKEQKYSF LQNPQTSLCF
SESIPTPSNR EETQQKSNLE LLRISLLLIQ SWLEPVQFLR
SVFANSLVYG ASDSNVYDLL KDLE

can be encoded by a synthetic gene taking account of optimal codon usage for the expression system. Synthetic genes are readily produced by total gene synthesis. For example the following sequence (producing CP_{—}01) could be constructed for expression in E. coli under control of a suitable promoter (ribosomal binding site underlined:

| 1 | GAGGACTTAAATTAAATA | |
|---|---|---|
| | Met Ile Gln Thr Leu Met Gly Arg Leu Glu Asp Gly Ser Pro Arg Thr Gly | 17 |
| 19 | ATG ATC CAG ACC TTA ATG GGC CTG CTG GAA GAC GGT AGC CCG CGG ACG GGA | |
| | Gln Ile Phe Lys Gln Thr Tyr Ser Lys Phe Asp Thr Asn Ser His Asn Asp | 34 |
| 70 | CAA ATT TTC AAA CAG ACC TAT AGT AAA TTT GAT ACG AAC AGC CAT AAC GAC | |
| | Asp Ala Leu Leu Lys Asn Tyr Gly Leu Leu Tyr Cys Phe Arg Lys Asp Met | 51 |
| 121 | GAT GCT CTA CTG AAA AAC TAT GGT CTG CTC TAC TGC TTC CGC AAG GAT ATG | |
| | Asp Lys Val Glu Thr Phe Leu Arg Ile Val Gln Cys Arg Ser Val Glu Gly | 68 |
| 172 | GAT AAA GTT GAA ACC TTT CTG CGC ATA GTG CAG TGT CGA TCT GTG GAG GGC | |
| | Ser Thr Ile Pro Leu Ser Arg Leu Phe Asp Asn Ala Ser Leu Arg Ala His | 85 |
| 223 | TCC ACT ATT CCT CTG TCC CGC TTG TTC GAC AAT GCC TCA TTA CGT GCA CAC | |
| | Arg Leu His Gln Leu Ala Phe Asp Thr Tyr Gln Glu Phe Glu Glu Ala Tyr | 102 |
| 274 | AGA TTG CAT CAG CTT GCC TTT GAT ACG TAC CAG GAG TTT GAA GAA GCG TAT | |
| | Ile Pro Lys Glu Gln Lys Tyr Ser Phe Leu Gln Asn Pro Gln Thr Ser Leu | 119 |
| 325 | ATT CCG AAG GAG CAA AAA TAC TCT TTT CTG CAA AAT CCG CAG ACC TCG CTG | |
| | Cys Phe Ser Glu Ser Ile Pro Thr Pro Ser Asn Arg Glu Glu Thr Gln Gln | 136 |
| 376 | TGC TTC AGT GAA AGC ATT CCG ACT CCA TCG AAC CGT GAG GAA ACA CAG CAG | |
| | Lys Ser Asn Leu Glu Leu Leu Arg Ile Ser Leu Leu Leu Ile Gln Ser Trp | 153 |
| 427 | AAA TCC AAT CTG GAA CTG CTT CGT ATC AGC TTA CTG CTC ATC CAA AGC TGG | |
| | Leu Glu Pro Val Gln Phe Leu Arg Ser Val Phe Ala Asn Ser Leu Val Tyr | 170 |
| 478 | TTG GAA CCC GTC CAA TTC CTT CGT TCA GTG TTT GCG AAT AGT CTG GTT TAT | |
| | Gly Ala Ser Asp Ser Asn Val Tyr Asp Leu Leu Lys Asp Leu Glu Stop | 185 |
| 529 | GGG GCA AGT GAC TCT AAC GTC TAT GAT CTG CTG AAA GAT CTC GAA TAA | |

This could be adapted to carry a suitable purification tag such as a polyhistidine tract incorporated at the C or N-terminus are required to aid in purification.

### EXAMPLE 2

An alternative permutation CP_{—}02 such as:

MEIQTLMGRL EDGSPRTGQI FKQTYSKFDT NSHNDDALLK
NYGLLYCFRK DMDKVETFLR IVQCRSVEGS TIPLSRLFDN
ASLRAHRLHQ LAFDTYQEFE EAYIPKEQKY SFLQNPQTSL
CFSESIPTPS NREETQQKSN LELLRISLLL IQSWLEPVQF
LRSVFANSLV YGASDSNVYD LLKDL

in which the break is made one residue earlier than in CP01

CP_O2 is encoded by:

| 1 | GAGGACTTAAATTAAATA | |
|---|---|---|
| | Met Glu Ile Gln Thr Leu Met Gly Arg Leu Glu Asp Gly Ser Pro Arg Thr | 17 |
| 19 | ATG GAA ATC CAG ACC TTA ATG GGC CGC CTG GAA GAC GGT AGC CCG CGG ACG | |
| | Gly Gln Ile Phe Lys Gln Thr Tyr Ser Lys Phe Asp Thr Asn Ser His Asn | 34 |
| 70 | GGA CAA ATT TTC AAA CAG ACC TAT AGT AAA TTT GAT ACG AAC AGC CAT AAC | |
| | Asp Asp Ala Leu Leu Lys Asn Tyr Gly Leu Leu Tyr Cys Phe Arg Lys Asp | 51 |
| 121 | GAC GAT GCT CTA CTG AAA AAC TAT GGT CTG CTC TAC TGC TTC CGC AAG GAT | |
| | Met Asp Lys Val Glu Thr Phe Leu Arg Ile Val Gln Cys Arg Ser Val Glu | 68 |
| 172 | ATG GAT AAA GTT GAA ACC TTT CTG CGC ATA GTG CAG TGT CGA TCT GTG GAG | |
| | gly Ser Thr Ile Pro Leu Ser Arg Leu Phe Asp Asn Ala Ser Leu Arg Ala | 85 |
| 223 | GGC TCC ACT ATT CCT CTG TCC CGC TTG TTC GAC AAT GCC TCA TTA CGT GCA | |
| | His Arg Leu His Gln Leu Ala Phe Asp Thr Tyr Gln Glu Phe Glu Glu Ala | 102 |
| 274 | CAC AGA TTG CAT CAG CTT GCC TTT GAT ACG TAC CAG GAG TTT GAA GAA GCG | |
| | Tyr Ile Pro Lys Glu Gln Lys Tyr Ser Phe Leu Gln Asn Pro Gln Thr Ser | 119 |
| 325 | TAT ATT CCG AAG GAG CAA AAA TAC TCT TTT CTG CAA AAT CCG CAG ACC TCG | |
| | Leu Cys Phe Ser Glu Ser Ile Pro Thr Pro Ser Asn Arg Glu Glu Thr Gln | 136 |
| 376 | CTG TGC TTC AGT GAA AGC ATT CCG ACT CCA TCG AAC CGT GAG GAA ACA CAG | |
| | Gln Lys Ser Asn Leu Glu Leu Leu Arg Ile Ser Leu Leu Leu Ile Gln Ser | 153 |
| 427 | CAG AAA TCC AAT CTG GAA CTG CTT CGT ATC AGC TTA CTG CTC ATC CAA AGC | |
| | Trp Leu Glu Pro Val Gln Phe Leu Arg Ser Val Phe Ala Asn Ser Leu Val | 170 |
| 478 | TGG TTG GAA CCC GTC CAA TTC CTT CGT TCA GTG TTT GCG AAT AGT CTG GTT | |
| | Tyr Gly Ala Ser Asp Ser Asn Val Tyr Asp Leu Leu Lys Asp Leu Stop | 185 |
| 529 | TAT GGG GCA AGT GAC TCT AAC GTC TAT GAT CTG CTG AAA GAT CTC TAA | |

### EXAMPLE 3

A further alternative, CP_{—}03

MQTLMGRLED GSPRTGQIFK QTYSKFDTNS HNDDALLKNY
GLLYCFRKDM DKVETFLRIV QCRSVEGSTI PLSRLFDNAS
LRAHRLHQLA FDTYQEFEEA YIPKEQKYSF LQNPQTSLCF
SESIPTPSNR EETQQKSNLE LLRISLLLIQ SWLEPVQFLR
SVFANSLVYG ASDSNVYDLL KDLE

in which the initial I in CP01 is removed and replaced by the M

Is encoded by:

| 1 | GAGGACTTAAATTAAATA | |
|---|---|---|
| | Met Gln Thr Leu Met Gly Arg Leu Glu Asp Gly Ser Pro Arg Thr Gly Gln | 17 |
| 19 | ATG CAG ACC TTA ATG GGC CGC CTG GAA GAC GGT AGC CCG CGG ACG GGA CAA | |
| | Ile Phe Lys Gln Thr Tyr Ser Lys Phe Asp Thr Asn Ser His Asn Asp Asp | 34 |
| 70 | ATT TTC AAA CAG ACC TAT AGT AAA TTT GAT ACG AAC AGC CAT AAC GAC GAT | |
| | Ala Leu Leu Lys Asn Tyr Gly Leu Leu Tyr Cys Phe Arg Lys Asp Met Asp | 51 |
| 121 | GCT CTA CTG AAA AAC TAT GGT CTG CTC TAC TGC TTC CGC AAG GAT ATG GAT | |
| | Lys Val Glu Thr Phe Leu Arg Ile Val Gln Cys Arg Ser Val Glu Gly Ser | 68 |
| 172 | AAA GTT GAA ACC TTT CTG CGC ATA GTG CAG TGT CGA TCT GTG GAG GGC TCC | |
| | Thr Ile Pro Leu Ser Arg Leu Phe Asp Asn Ala Ser Leu Arg Ala His Arg | 85 |
| 223 | ACT ATT CCT CTG TCC CGC TTG TTC GAC AAT GCC TCA TTA CGT GCA CAC AGA | |
| | Leu His Gln Leu Ala Phe Asp Thr Tyr Gln Glu Phe Glu Glu Ala Tyr Ile | 102 |
| 274 | TTG CAT CAG CTT GCC TTT GAT ACG TAC CAG GAG TTT GAA GAA GCG TAT ATT | |
| | Pro Lys Glu Gln Lys Tyr Ser Phe Leu Gln Asn Pro Gln Thr Ser Leu Cys | 119 |
| 325 | CCG AAG GAG CAA AAA TAC TCT TTT CTG CAA AAT CCG CAG ACC TCG CTG TGC | |
| | Phe Ser Glu Ser Ile Pro Thr Pro Ser Asn Arg Glu Glu Thr Gln Gln Lys | 136 |
| 376 | TTC AGT GAA AGC ATT CCG ACT CCA TCG AAC CGT GAG GAA ACA CAG CAG AAA | |
| | Ser Asn Leu Glu Leu Leu Arg Ile Ser Leu Leu Leu Ile Gln Ser Trp Leu | 153 |
| 427 | TCC AAT CTG GAA CTG CTT CGT ATC AGC TTA CTG CTC ATC CAA AGC TGG TTG | |
| | Glu Pro Val Gln Phe Leu Arg Ser Val Phe Ala Asn Ser Leu Val Tyr Gly | 170 |
| 478 | GAA CCC GTC CAA TTC CTT CGT TCA GTG TTT GCG AAT AGT CTG GTT TAT GGG | |
| | Ala Ser Asp Ser Asn Val Tyr Asp Leu Leu Lys Asp Leu Glu Stop | 184 |
| 529 | GCA AGT GAC TCT AAC GTC TAT GAT CTG CTG AAA GAT CTC GAA TAA | |

### EXAMPLE 4

A further variation: Position cysteine residues at C and N termini—so that they may form a disulphide bond, thus making a covalently closed, circular molecule:

CP_04
MCIQTLMGRL EDGSPRTGQI FKQTYSKFDT NSHNDDALLK
NYGLLYCFRK DMDKVETFLR IVQCRSVEGS TIPLSRLFDN
ASLRAHRLHQ LAFDTYQEFE EAYIPKEQKY SFLQNPQTSL
CFSESIPTPS NREETQQKSN LELLRISLLL IQSWLEPVQF
LRSVFANSLV YGASDSNVYD LLKDLEC

New cystein residues underlined.

Encoded by:

| 1 | GAGGACTTAAATTAAATA | |
|---|---|---|
| | Met Cys Ile Gln Thr Leu Met Gly Arg Leu Glu Asp Gly Ser Pro Arg Thr | 17 |
| 19 | ATG TGT ATC CAG ACC TTA ATG GGC CGC CTG GAA GAC GGT AGC CCG CGG ACG | |
| | Gly Gln Ile Phe Lys Gln Thr Tyr Ser Lys Phe Asp Thr Asn Ser His Asn | 34 |
| 70 | GGA CAA ATT TTC AAA CAG ACC TAT AGT AAA TTT GAT ACG AAC AGC CAT AAC | |
| | Asp Asp Ala Leu Leu Lys Asn Tyr Gly Leu Leu Tyr Cys Phe Arg Lys Asp | 51 |
| 121 | GAC GAT GCT CTA CTG AAA AAC TAT GGT CTG CTC TAC TGC TTC CGC AAG GAT | |
| | Met Asp Lys Val Glu Thr Phe Leu Arg Ile Val Gln Cys Arg Ser Val Glu | 68 |
| 172 | ATG GAT AAA GTT GAA ACC TTT CTG CGC ATA GTG CAG TGT CGA TCT GTG GAG | |
| | Gly Ser Thr Ile Pro Leu Ser Arg Leu Phe Asp Asn Ala Ser Leu Arg Ala | 85 |
| 223 | GGC TCC ACT ATT CCT CTG TCC CGC TTG TTC GAC AAT GCC TCA TTA CGT GCA | |
| | His Arg Leu His Gln Leu Ala Phe Asp Thr Tyr Gln Glu Phe Glu Glu Ala | 102 |
| 274 | CAC AGA TTG CAT CAG CTT GCC TTT GAT ACG TAC CAG GAG TTT GAA GAA GCG | |
| | Tyr Ile Pro Lye Glu Gln Lys Tyr Ser Phe Leu Gln Asn Pro Gln Thr Ser | 119 |
| 325 | TAT ATT CCG AAG GAG CAA AAA TAC TCT TTT CTG CAA AAT CCG CAG ACC TCG | |
| | Leu Cys Phe Ser Glu Ser Ile Pro Thr Pro Ser Asn Arg Glu Glu Thr Gln | 136 |
| 376 | CTG TGC TTC AGT GAA AGC ATT CCG ACT CCA TCG AAC CGT GAG GAA ACA CAG | |
| | Gln Lys Ser Asn Leu Glu Leu Leu Arg Ile Ser Leu Leu Leu Ile Gln Ser | 153 |
| 427 | CAG AAA TCC AAT CTG GAA CTG CTT CGT ATC AGC TTA CTG CTC ATC CAA AGC | |
| | Trp Leu Glu Pro Val Gln Phe Leu Arg Ser Val Phe Ala Asn Ser Leu Val | 170 |
| 478 | TGG TTG GAA CCC GTC CAA TTC CTT CGT TCA GTG TTT GCG AAT AGT CTG GTT | |
| | Tyr Gly Ala Ser Aep Ser Asn Val Tyr Asp Leu Leu Lys Asp Leu Glu Cys | 187 |
| 529 | TAT GGG GCA AGT GAC TCT AAC GTC TAT GAT GTG CTG AAA GAT CTC GAA TGT | |
| | Stop | 187 |
| 580 | TAA | |

1. A modified cytokine ligand polypeptide comprising a modified amino acid sequence which is a modification of the native cytokine amino acid sequence of said ligand, wherein the native amino terminal and carboxyl terminal amino acid residues of the native polypeptide are linked, directly or indirectly, together, characterised in that said ligand is provided with alternative amino terminal and carboxyl terminal amino acid residues and further wherein at least one binding domain for said ligand's cognate binding partner is disrupted.

2. A modified cytokine ligand polypeptide according to claim 1 wherein said ligand is selected from the group consisting of: growth hormone; leptin; erythropoietin; prolactin; tumour necrosis factor (TNF), interleukins (IL), IL-2, IL-3, IL-4, IL-5, IL-6, IL-7, IL-9, IL-10, IL-11; the p35 subunit of IL-12, IL-13, IL-15; granulocyte colony stimulating factor (G-CSF); granulocyte macrophage colony stimulating factor (GM-CSF); ciliary neurotrophic factor (CNTF); cardiotrophin-1 (CT-1); leukemia inhibitory factor (LIF); oncostatin M (OSM); interferon, IFNα and IFNγ, osteoprotogerin (OPG).

3. A modified cytokine ligand polypeptide according to claim 2 wherein said ligand is growth hormone.

4. A modified cytokine ligand polypeptide according to claim 1 wherein said native amino terminal and carboxyl terminal amino acid residues are directly linked to each other.

5. A modified cytokine ligand polypeptide according to claim 1 wherein said native amino terminal and carboxyl terminal amino acid residues are indirectly linked by a linking molecule.

6. A modified cytokine ligand polypeptide according to claim 5 wherein said linking molecule is a peptide linker.

7. A modified cytokine ligand polypeptide according to claim 6 wherein said linking peptide is a flexible peptide linker.

8. A modified cytokine ligand polypeptide according to claim 7 wherein said flexible linker is a polypeptide which comprises 5 to 30 amino acid residues.

9. A modified cytokine ligand polypeptide according to claim 8 wherein the linker comprises 10 to 20 amino acid residues.

10. A mollified cytokine ligand polypeptide according to claim 6 wherein said linker comprises at least one copy of the peptide: Gly Gly Gly Gly Ser.

11. A modified cytokine ligand polypeptide according to claim 5 wherein said linker is an inflexible linker.

12. A modified cytokine ligand polypeptide according to claim 11 wherein said linker comprises a α-helical region.

13. A modified cytokine ligand polypeptide according to claim 1 wherein a receptor binding domain of said ligand comprises a low affinity bind site.

14. A modified cytokine ligand polypeptide according to claim 13 wherein said low affinity binding domain is site 2 of growth hormone.

15. A modified cytokine ligand polypeptide according to claim 14 wherein said low affinity binding domain of growth hormone is between about amino acid 116 to amino acid 122 of human growth hormone as represented by the amino acid sequence shown in FIG. 1.

16. A modified cytokine ligand polypeptide according to claim 15 wherein the alternative amino terminal and carboxyl terminal amino acid residues are derived from between amino acid 116 and amino acid 122 of human growth hormone as represented by FIG. 1.

17. A modified cytokine ligand polypeptide according to claim 16 wherein said alternative amino terminal and carboxyl terminal amino acid residues are derived from between amino acid 118 and amino acid 121 of human growth hormone as represented by FIG. 1.

18. A modified cytokine ligand polypeptide according to claim 16 wherein said alternative amino terminal and carboxyl terminal amino acid residues are derived from between amino acid 119 and amino acid 121 of human growth hormone as represented by FIG. 1.

19. A modified cytokine ligand polypeptide according to claim 16 wherein said alternative amino terminal and carboxyl terminal amino acid residues are derived from between amino acid 120 and amino acid 121 of human growth hormone as represented by FIG. 1.

20. A modified cytokine ligand polypeptide according to claim 16 wherein said alternative amino terminal and carboxyl terminal amino acid residues are derived from between amino acid 118 and amino acid 120 of human growth hormone as represented by FIG. 1.

21. A modified cytokine ligand polypeptide to claim 16 wherein said alternative amino terminal and carboxyl terminal amino acid residues are derived from between amino acid 119 and amino acid 120 of human growth hormone as represented by FIG. 1.

22. A modified cytokine ligand polypeptide according to claim 14 wherein said alternative amino terminal and carboxyl terminal amino acid residues are derived from between about amino acid 100 and amino acid 102 of human growth hormone as represented by the amino acid sequence shown in FIG. 1.

23. A modified cytokine ligand polypeptide according to claim 14 wherein said alternative amino terminal and carboxyl terminal amino acid residues are derived from between about amino acid 130 and amino acid 132 of human growth hormone as represented by the amino acid sequence shown in FIG. 1.

24. An oligomeric cytokine ligand polypeptide comprising at least two modified cytokine ligand polypeptides according to claim 1, wherein said ligands are linked, either directly or indirectly, together.

25. An oligomeric cytokine ligand according to claim 24 wherein said polypeptides are linked by a peptide linker comprising a α-helical region.

26. An oligomeric cytokine ligand according to claim 24 wherein said oligomer comprises two modified cytokine ligand polypeptides.

27. An oligomeric cytokine ligand according to claim 26 wherein said oligomer comprises, at least 3 modified cytokine ligand polypeptides.

28. An oligomeric cytokine ligand according to claim 26 wherein said oligomer comprises at least two modified growth hormone polypeptides.

29. An oligomeric cytokine ligand according to claim 28 wherein said oligomeric growth hormone polypeptide comprises multiple ligand polypeptides.

30. An oligomeric cytokine ligand according to claim 24
comprising at least one modified cytokine ligand polypeptide according to claim 1 linked, either directly or indirectly, to at least one native cytokine ligand polypeptide from which said modified cytokine ligand polypeptide was derived.

31. An oligomeric cytokine ligand according to claim 24 wherein said modified cytokine ligand polypeptide according to claim 1 is linked to the extracellular ligand binding domain of said ligands cognate receptor.

32. An oligomeric cytokine ligand according to claim 24 wherein said linker comprises a cleavage site.

33. An oligomeric cytokine ligand according to claim 32 wherein said cleavage site is a proteolytic cleavage site.

34. An oligomeric cytokine ligand according to claim 33 wherein said cleavage site is sensitive to a serum protease

35. An oligomeric cytokine ligand according to claim 33 wherein said cleavage site comprises the amino acid sequence: LVPRGS, or functional variant thereof.

36. An oligomeric cytokine ligand according to claim 33 wherein said cleavage site comprises at least one copy of the amino acid sequence: GGGGS, or functional variant thereof.

37. An oligomeric cytokine ligand according to claim 36 wherein said cleavage site comprises the amino acid sequence PGI(S).

38. An oligomeric cytokine ligand according to claim 33 wherein said cleavage site comprises the amino acid sequence: LVGPRGSPGI.

39. An oligomeric cytokine ligand according to claim 36 wherein said cleavage site comprises at least two copies of the amino acid sequence GGGGS that flank said cleavage site.

40. An oligomeric cytokine ligand according to claim 39 wherein said cleavage site is sensitive to the serum protease thrombin.

41. An isolated nucleic acid molecule which encodes a modified cytokine ligand polypeptide according to claim 1 or an oligomeric modified cytokine ligand polypeptide comprising at least two modified cytokine ligand polypeptides according to claim 1, wherein said ligands are linked, either directly or indirectly, together.

42. A vector comprising the nucleic acid molecule according to claim 41.

43. A host cell transfected or transformed with a nucleic acid molecule according to claim 41 or a vector comprising the nucleic acid molecule of claim 41.

44. A host cell according to claim 43 wherein said cell is a eukaryotic cell.

45. A host cell according to claim 44 wherein said cell is a mammalian cell, a yeast cell, an insect cell, or a plant cell.

46. A host cell according to claim 43 wherein said cell is a prokaryotic cell.

47. A non-human transgenic mammal transfected or transformed with the nucleic acid molecule according to claim 41 or a vector comprising the nucleic acid molecule according to claim 41.

48. A pharmaceutical composition comprising the modified cytokine ligand polypeptide of claim 1, an oligomeric form thereof, ptide, a nucleic acid molecule encoding the modified cytokine ligand polypeptide or oligomeric form thereof, a vector comprising the nucleic acid molecule encoding the modified cytokine ligand polypeptide or oligomeric form thereof, or a cell transformed with the nucleic acid molecule or the vector, in a pharmaceutically acceptable carrier.

49. A screening method to generate modified cytokine ligand polypeptides according to claim 1 comprising the steps of:
i) forming a preparation comprising native cytokine ligand polypeptide molecules wherein the native amino terminal and carboxyl terminal amino acids are linked either directly or indirectly together;
ii) generating modified cytokine ligand polypeptide molecules wherein said molecules have alternative amino terminal and carboxyl terminal amino acids; and
iii) testing the activity of said modified cytokine ligand polypeptides.

50. A method according to claim 49 wherein said native cytokine is growth hormone.

51. A modified cytokine ligand polypeptide identified by the method according to claim 49.

52. A ligand according to claim 51 wherein said modified cytokine ligand polypeptide is growth hormone.

53. A method of treatment of subject comprising administering an effective amount the modified cytokine ligand polypeptide of claim 1, an oligomeric form thereof, a nucleic acid molecule encoding the modified cytokine ligand polypeptide or oligomeric form thereof, a vector comprising the nucleic acid molecule encoding the modified cytoke ligand polypeptide or oligomeric form thereof, a cell transformed with the nucleic acid molecule or the vector, or a combination thereof.

54. A modified cytokine ligand polypeptide according to claim 17 wherein said alternative amino terminal and carboxyl terminal amino acid residues are derived from between amino acid 119 and amino acid 121 of human growth hormone as represented by FIG. 1.

55. A modified cytokine ligand polypeptide according to claim 17 wherein said alternative amino terminal and carboxyl terminal amino acid residues are derived from between amino acid 120 and amino acid 121 of human growth hormone as represented by FIG. 1.

56. A modified cytokine ligand polypeptide according to claim 17 wherein said alternative amino terminal and carboxyl terminal amino acid residues are derived from between amino acid 118 and amino acid 120 of human growth hormone as represented by FIG. 1.

57. A modified cytokine ligand polypeptide to claim 17 wherein said alternative amino terminal and carboxyl terminal amino acid residues are derived from between amino acid 119 and amino acid 120 of human growth hormone as represented by FIG. 1.


SEQUENCE LISTING (32 sequences)

SEQ ID NO: 1 - protein, 184 aa, Homo sapiens
IQTLMGRLEDGSPRTGQIFKQTYSKFDTNSHNDDALLKNYGLLYCFRKDMDKVETFLRIVQCRSVEGSTIPLSRLFDNASLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLE

SEQ ID NO: 2 - DNA, 576 bp, Homo sapiens
gaggacttaaattaaataatgatccagaccttaatgggccgcctggaagacggtagcccgcggacgggacaaattttcaaacagacctatagtaaatttgatacgaacagccataacgacgatgctctactgaaaaactatggtctgctctactgcttccgcaaggatatggataaagttgaaacctttctgcgcatagtgcagtgtcgatctgtggagggctccactattcctctgtcccgcttgttcgacaatgcctcattacgtgcacacagattgcatcagcttgcctttgatacgtaccaggagtttgaagaagcgtatattccgaaggagcaaaaatactcttttctgcaaaatccgcagacctcgctgtgcttcagtgaaagcattccgactccatcgaaccgtgaggaaacacagcagaaatccaatctggaactgcttcgtatcagcttactgctcatccaaagctggttggaacccgtccaattccttcgttcagtgtttgcgaatagtctggtttatggggcaagtgactctaacgtctatgatctgctgaaagatctcgaataa

SEQ ID NO: 3 - protein, 185 aa, Homo sapiens
MEIQTLMGRLEDGSPRTGQIFKQTYSKFDTNSHNDDALLKNYGLLYCFRKDMDKVETFLRIVQCRSVEGSTIPLSRLFDNASLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDL

SEQ ID NO: 4 - DNA, 576 bp, Homo sapiens
gaggacttaaattaaataatggaaatccagaccttaatgggccgcctggaagacggtagcccgcggacgggacaaattttcaaacagacctatagtaaatttgatacgaacagccataacgacgatgctctactgaaaaactatggtctgctctactgcttccgcaaggatatggataaagttgaaacctttctgcgcatagtgcagtgtcgatctgtggagggctccactattcctctgtcccgcttgttcgacaatgcctcattacgtgcacacagattgcatcagcttgcctttgatacgtaccaggagtttgaagaagcgtatattccgaaggagcaaaaatactcttttctgcaaaatccgcagacctcgctgtgcttcagtgaaagcattccgactccatcgaaccgtgaggaaacacagcagaaatccaatctggaactgcttcgtatcagcttactgctcatccaaagctggttggaacccgtccaattccttcgttcagtgtttgcgaatagtctggtttatggggcaagtgactctaacgtctatgatctgctgaaagatctctaa

SEQ ID NO: 5 - protein, 184 aa, Homo sapiens
MQTLMGRLEDGSPRTGQIFKQTYSKFDTNSHNDDALLKNYGLLYCFRKDMDKVETFLRIVQCRSVEGSTIPLSRLFDNASLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLE

SEQ ID NO: 6 - DNA, 573 bp, Homo sapiens
gaggacttaaattaaataatgcagaccttaatgggccgcctggaagacggtagcccgcggacgggacaaattttcaaacagacctatagtaaatttgatacgaacagccataacgacgatgctctactgaaaaactatggtctgctctactgcttccgcaaggatatggataaagttgaaacctttctgcgcatagtgcagtgtcgatctgtggagggctccactattcctctgtcccgcttgttcgacaatgcctcattacgtgcacacagattgcatcagcttgcctttgatacgtaccaggagtttgaagaagcgtatattccgaaggagcaaaaatactcttttctgcaaaatccgcagacctcgctgtgcttcagtgaaagcattccgactccatcgaaccgtgaggaaacacagcagaaatccaatctggaactgcttcgtatcagcttactgctcatccaaagctggttggaacccgtccaattccttcgttcagtgtttgcgaatagtctggtttatggggcaagtgactctaacgtctatgatctgctgaaagatctcgaataa

SEQ ID NO: 7 - protein, 187 aa, Homo sapiens
MCIQTLMGRLEDGSPRTGQIFKQTYSKFDTNSHNDDALLKNYGLLYCFRKDMDKVETFLRIVQCRSVEGSTIPLSRLFDNASLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEC

SEQ ID NO: 8 - DNA, 582 bp, Homo sapiens
gaggacttaaattaaataatgtgtatccagaccttaatgggccgcctggaagacggtagcccgcggacgggacaaattttcaaacagacctatagtaaatttgatacgaacagccataacgacgatgctctactgaaaaactatggtctgctctactgcttccgcaaggatatggataaagttgaaacctttctgcgcatagtgcagtgtcgatctgtggagggctccactattcctctgtcccgcttgttcgacaatgcctcattacgtgcacacagattgcatcagcttgcctttgatacgtaccaggagtttgaagaagcgtatattccgaaggagcaaaaatactcttttctgcaaaatccgcagacctcgctgtgcttcagtgaaagcattccgactccatcgaaccgtgaggaaacacagcagaaatccaatctggaactgcttcgtatcagcttactgctcatccaaagctggttggaacccgtccaattccttcgttcagtgtttgcgaatagtctggtttatggggcaagtgactctaacgtctatgatctgctgaaagatctcgaatgttaa

SEQ ID NO: 9 - protein, 191 aa, Homo sapiens
FPTIPLSRLFDNASLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 10 - DNA, 573 bp, Homo sapiens
ttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttc

SEQ ID NO: 11 - protein, 638 aa, Homo sapiens
MDLWQLLLTLALAGSSDAFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFYFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 12 - DNA, 552 bp, Homo sapiens
atccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagcaccattcccttatccaggctttttgacaacgctagtctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagag

SEQ ID NO: 13 - DNA, 33 bp, Homo sapiens
gctaggatccaacccttatccaaacgctgatgg

SEQ ID NO: 14 - DNA, 32 bp, Homo sapiens
tggataagggaatggtgctgccctccacagag

SEQ ID NO: 15 - DNA, 39 bp, Homo sapiens
gtcaactggtcagcggccgccctctaggtcctttaggag

SEQ ID NO: 16 - DNA, 32 bp, Homo sapiens
ctctgtggagggcagcaccattcccttatcca

SEQ ID NO: 17 - DNA, 32 bp, Homo sapiens; identical to SEQ ID NO: 14

SEQ ID NO: 18 - DNA, 33 bp, Homo sapiens; identical to SEQ ID NO: 13

SEQ ID NO: 19 - DNA, 39 bp, Homo sapiens; identical to SEQ ID NO: 15

SEQ ID NO: 20 - DNA, 33 bp, Homo sapiens; identical to SEQ ID NO: 13

SEQ ID NO: 21 - DNA, 40 bp, Homo sapiens
gtcaactggtcagcggccgccttcctctaggtcctttagg

SEQ ID NO: 22 - DNA, 33 bp, Homo sapiens; identical to SEQ ID NO: 13

SEQ ID NO: 23 - DNA, 36 bp, Homo sapiens
gctaggatccaacccttggcatccaaacgctgatgg

SEQ ID NO: 24 - DNA, 36 bp, Homo sapiens; identical to SEQ ID NO: 23

SEQ ID NO: 25 - DNA, 39 bp, Homo sapiens; identical to SEQ ID NO: 15

SEQ ID NO: 26 - DNA, 36 bp, Homo sapiens; identical to SEQ ID NO: 23

SEQ ID NO: 27 - DNA, 40 bp, Homo sapiens; identical to SEQ ID NO: 21

SEQ ID NO: 28 - DNA, 38 bp, Homo sapiens
gctaggatccaacccttgtgtacggcgcctctgacagc

SEQ ID NO: 29 - DNA, 41 bp, Homo sapiens
gtcaactggtcagcggccgccgctgttggcgaagacactcc

SEQ ID NO: 30 - DNA, 35 bp, Homo sapiens
gctaggatccaacccttagcccccggactgggcag

SEQ ID NO: 31 - DNA, 40 bp, Homo sapiens
gtcaactggtcagcggccgccatcttccagcctccccatc

SEQ ID NO: 32 - protein, 185 aa, Homo sapiens
MIQTLMGRLENGSPRTGQIFKQTYSKFDTNSHNDDALLKNYGLLYCFRKDMDKVETFLRIVQCRSVEGSTIPLSRLFDNASLRAHRLHQLAFDTYQEFEEAYIPKEQKTSFLQNPQTSLCFSESIPTPSNREETQQKSNLELLRISLLLIQSWLEPVQFLRSVFANSLVYGASDSNVYDLLKDLE